SEQUENCE LISTING (25 sequences)

SEQ ID NO: 1 - protein, 8 aa, synthetic construct
  REGION 1..8: note = Description of sequence: streptavidin binding cyclic peptide
  DISULFID 2..8: note = CIS-CIS Bond
  REGION 2..8: note = These aminos acids are cyclic
CHPQGPPC

SEQ ID NO: 2 - protein, 8 aa, synthetic construct
  REGION 1..8: note = Description of sequence: streptavidin binding cyclic peptide
  DISULFID 2..8: note = CIS-CIS Bond
  REGION 2..8: note = These aminos acids are cyclic
CHPQFPLC

SEQ ID NO: 3 - protein, 8 aa, synthetic construct
  REGION 1..8: note = Description of sequence: streptavidin binding cyclic peptide
  DISULFID 2..8: note = CIS-CIS Bond
  REGION 2..8: note = These aminos acids are cyclic
CHPQFTLC

SEQ ID NO: 4 - protein, 8 aa, synthetic construct
  REGION 1..8: note = Description of sequence: streptavidin binding cyclic peptide
  DISULFID 2..8: note = CIS-CIS Bond
  REGION 2..8: note = These aminos acids are cyclic
CHPQFNLC

SEQ ID NO: 5 - protein, 8 aa, synthetic construct
  REGION 1..8: note = Description of sequence: streptavidin binding cyclic peptide
  DISULFID 2..8: note = CIS-CIS Bond
  REGION 2..8: note = These aminos acids are cyclic
CHPQGDRC

SEQ ID NO: 6 - protein, 8 aa, synthetic construct
  REGION 1..8: note = Description of sequence: streptavidin binding cyclic peptide
  DISULFID 2..8: note = CIS-CIS Bond
  REGION 2..8: note = These aminos acids are cyclic
CHPQFSNC

SEQ ID NO: 7 - protein, 8 aa, synthetic construct
  REGION 1..8: note = Description of sequence: streptavidin binding cyclic peptide
  DISULFID 2..8: note = CIS-CIS Bond
  REGION 2..8: note = These aminos acids are cyclic
CHPQFRHC

SEQ ID NO: 8 - protein, 8 aa, synthetic construct
  REGION 1..8: note = Description of sequence: streptavidin binding cyclic peptide
  DISULFID 2..8: note = CIS-CIS Bond
  REGION 2..8: note = These aminos acids are cyclic
CHPQSGRC

SEQ ID NO: 9 - protein, 7 aa, synthetic construct
  REGION 1..7: note = Description of sequence: streptavidin binding cyclic peptide
  DISULFID 2..7: note = CIS-CIS Bond
  REGION 2..7: note = These aminos acids are cyclic
CHPQFPC

SEQ ID NO: 10 - protein, 7 aa, synthetic construct
  REGION 1..7: note = Description of sequence: streptavidin binding cyclic peptide
  DISULFID 2..7: note = CIS-CIS Bond
  REGION 2..7: note = These aminos acids are cyclic
CHPQVPC

SEQ ID NO: 11 - protein, 7 aa, synthetic construct
  REGION 1..7: note = Description of sequence: streptavidin binding cyclic peptide
  DISULFID 2..7: note = CIS-CIS Bond
  REGION 2..7: note = These aminos acids are cyclic
CHPQFNC

SEQ ID NO: 12 - protein, 7 aa, synthetic construct
  REGION 1..7: note = Description of sequence: streptavidin binding cyclic peptide
  DISULFID 2..7: note = CIS-CIS Bond
  REGION 2..7: note = These aminos acids are cyclic
CHPQVAC

SEQ ID NO: 13 - protein, 7 aa, synthetic construct
  REGION 1..7: note = Description of sequence: streptavidin binding cyclic peptide
  DISULFID 2..7: note = CIS-CIS Bond
  REGION 2..7: note = These aminos acids are cyclic
CHPQFMC

SEQ ID NO: 14 - protein, 7 aa, synthetic construct
  REGION 1..7: note = Description of sequence: streptavidin binding cyclic peptide
  DISULFID 2..7: note = CIS-CIS Bond
  REGION 2..7: note = These aminos acids are cyclic
CHPQFRC

SEQ ID NO: 15 - protein, 7 aa, synthetic construct
  REGION 1..7: note = Description of sequence: streptavidin binding cyclic peptide
  DISULFID 2..7: note = CIS-CIS Bond
  REGION 2..7: note = These aminos acids are cyclic
CHPQNAC

SEQ ID NO: 16 - protein, 7 aa, synthetic construct
  REGION 1..7: note = Description of sequence: streptavidin binding cyclic peptide
  DISULFID 2..7: note = CIS-CIS Bond
  REGION 2..7: note = These aminos acids are cyclic
CHPQVSC

SEQ ID NO: 17 - protein, 7 aa, synthetic construct
  REGION 1..7: note = Description of sequence: streptavidin binding cyclic peptide
  DISULFID 2..7: note = CIS-CIS Bond
  REGION 2..7: note = These aminos acids are cyclic
CHPQFAC

SEQ ID NO: 18 - protein, 7 aa, synthetic construct
  REGION 1..7: note = Description of sequence: streptavidin binding cyclic peptide
  DISULFID 2..7: note = CIS-CIS Bond
  REGION 2..7: note = These aminos acids are cyclic
CHPQVRC

SEQ ID NO: 19 - protein, 7 aa, synthetic construct
  REGION 1..7: note = Description of sequence: streptavidin binding cyclic peptide
  DISULFID 2..7: note = CIS-CIS Bond
  REGION 2..7: note = These aminos acids are cyclic
CWHPQFC

SEQ ID NO: 20 - protein, 6 aa, synthetic construct
  REGION 1..6: note = Description of sequence: streptavidin binding cyclic peptide
  DISULFID 2..6: note = CIS-CIS Bond
  REGION 2..6: note = These aminos acids are cyclic
CHPQFC

SEQ ID NO: 21 - protein, 27 aa, synthetic construct
  REGION 1..27: note = Description of sequence: cyclic polypeptide
  DISULFID 11..18: note = CIS-CIS Bond
  REGION 11..18: note = These aminos acids are cyclic
GGGGSGGGGSCHPQPFCGGGGSGGGGS

SEQ ID NO: 22 - protein, 7 aa, synthetic construct
  REGION 1..7: note = Description of sequence: streptavidin binding cyclic peptide
  DISULFID 1..7: note = CIS-CIS Bond
  REGION 1..7: note = These aminos cyclic
CHPQPFC

SEQ ID NO: 23 - protein, 7 aa, synthetic construct
  REGION 1..7: note = Description of sequence: Linker peptide control
GHPQPFG

SEQ ID NO: 24 - protein, 5 aa, synthetic construct
  REGION 1..5: note = Description of sequence: VH mutant
IANVA

SEQ ID NO: 25 - protein, 5 aa, synthetic construct
  REGION 1..5: note = Description of sequence: VH mutant
IAGVA

(19) United States
(12) Patent Application Publication
(10) Pub. No.: US 2026/0022187 A1
(43) Pub. Date: Jan. 22, 2026

(54) METHODS AND COMPOSITIONS REGARDING CYCLIC PEPTIDES FOR USE WITH ANTIBODIES AND ANTIBODY FRAGMENTS

(71) Applicant: OHIO STATE INNOVATION FOUNDATION, Columbus, OH (US)

(72) Inventors: Thomas MAGLIERY, Columbus, OH (US); Jeong Min HAN, Gaithersburg, MD (US); Zirui ZHU, Columbus, OH (US); To Uyen DO, Chapel Hill, NC (US)

(21) Appl. No.: 18/995,612

(22) PCT Filed: Jul. 28, 2023
(86) PCT No.: PCT/US2023/071232

§ 371 Date: Jan. 16, 2025

Related U.S. Application Data
(60) Provisional application No. 63/393,112, filed on Jul. 28, 2022.

(51) Int. Cl. C07K 16/28; A61K 38/00; C07K 7/64; C07K 14/00

(52) U.S. Cl. CPC C07K 16/2896; C07K 7/64; C07K 14/001; A61K 38/00; C07K 2317/31; C07K 2317/622; C07K 2317/626; C07K 2319/22

(57) ABSTRACT

The present disclosure relates to methods and compositions regarding cyclic peptides for use with antibodies or fragments thereof.

### RELATED APPLICATION

This application claims priority to, and the benefit of, U.S. Provisional Patent Application No. 63/393,112, filed Jul. 28, 2022, entitled “METHODS AND COMPOSITIONS REGARDING CYCLIC PEPTIDES FOR USE WITH ANTIBODIES AND ANTIBODY FRAGMENTS,” which is incorporated by reference herein in its entirety.

### REFERENCE TO SEQUENCE LISTING

The sequence listing submitted on Jul. 28, 2023, as an.XML file entitled “103361-336WO1_ST26.xml” created on Jul. 25, 2023, and having a file size of 49,765 bytes is hereby incorporated by reference pursuant to 37 C.F.R. § 1.52(e)(5).

### FIELD

The present disclosure relates to methods and compositions regarding cyclic peptides for use with antibodies or fragments thereof.

### BACKGROUND

Antibodies are integral parts of the immune system that recognize specific foreign molecules to trigger the adaptive immune responses. Due to the high selectivity and high binding affinity of antibodies, they have been used for applications such as detecting tumor markers or selectively delivering antitumor compounds to cancer cells. These exciting applications motivate the design of antibodies that can target molecules inside the cell.

Cyclic peptides are polypeptide chains which contain a circular sequence of bonds. This can be through a connection between the amino and carboxyl ends of the peptide, for example in cyclosporin; a connection between the amino end and a side chain, for example in bacitracin; the carboxyl end and a side chain, for example in colistin; or two side chains or more complicated arrangements, for example in amanitin. Many cyclic peptides have been discovered in nature and many others have been synthesized in the laboratory. In nature, they are frequently antimicrobial or toxic; in medicine they have various applications, for example as antibiotics and immunosuppressive agents.

The moderate size and diverse functional groups of peptides ensure that the contact area is large enough to provide high selectivity, and the formation of multiple hydrogen bonds can lead to strong binding affinity. In addition, cyclization of peptides generates structural and functional features that are critical for their use as pharmaceutical agents. The structural constraints provided by cyclization help to resist degradation by proteases in the blood, thereby increasing their serum stability. Cyclization of peptides also facilitates passage through the cell membrane, thus broadening the use of cyclic peptides beyond extracellular targets to include intracellular targets.

Given the numerous applications described above, there is a need in the art for cyclized peptides which can perform a variety of useful functions in both therapeutic and diagnostic applications.

### SUMMARY

The present disclosure provides synthetic cyclic peptide compositions and methods of use thereof.

In one aspect, disclosed herein is a peptide-antibody complex comprising a cyclic polypeptide operably linked to an antibody or fragments thereof, wherein the cyclic polypeptide comprises an amino acid sequence of X-Cys-Y-Cys-Z, wherein X is an amino acid sequence of 0-30 amino acids, Y is an amino acid sequence of 2-20 amino acids, Z is an amino acid sequence of 0-30 amino acids, and at least two Cys are cysteine amino acids; and wherein the antibody or fragments thereof comprise a single chain variable fragment (scFv).

In some embodiments, the at least two Cys form a disulfide bridge. In some embodiments, the scFv comprises a heavy chain and a light chain. In some embodiments, the antibody or fragments thereof comprises a bispecific antibody. In some embodiments, the antibody or fragments thereof are operably linked to a C terminus, a N terminus, or both C and N termini of the cyclic polypeptide.

In some embodiments, the cyclic polypeptide becomes a linker when the antibody or fragments thereof are on both the N and C terminus of the cyclic polypeptide. In some embodiments, the cyclic polypeptide becomes a linker when the heavy chain of the scFv is on one side of the linker and the light chain of the scFv is on another side of the linker.

In some embodiments, the scFv comprises a diabody or a tetrabody.

In some embodiments, the cyclic polypeptide comprises 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO: 21. In some embodiments, the cyclic polypeptide comprises SEQ ID NO: 21.

In some embodiments, the disulfide bridge forms a loop or a circle within the complex. In some embodiments, the loop or the circle form spontaneously.

In one aspect, disclosed herein is a synthetic nucleic acid encoding the cyclic polypeptide of any preceding aspect.

In one aspect, disclosed herein is a vector comprising the synthetic nucleic acid of any preceding aspect.

In one aspect, disclosed herein is a cell comprising the vector of any preceding aspect.

In one aspect, disclosed herein is a method of treating a subject in need of therapeutic intervention, the method comprising administering to the subject a peptide-antibody complex comprising a cyclic polypeptide operably linked to an antibody or fragments thereof, wherein the cyclic polypeptide comprises an amino acid sequence of X-Cys-Y-Cys-Z, wherein X is an amino acid sequence of 0-30 amino acids, Y is an amino acid sequence of 2-20 amino acids, Z is an amino acid sequence of 0-30 amino acids, and at least two Cys are cysteine amino acids; wherein the antibody or fragments thereof comprise a single chain variable fragment (scFv); and wherein the cyclic polypeptide improves binding of the antibody or fragments to an antigen.

In one aspect, disclosed herein is a method of diagnosing a subject with a disease or disorder, the method comprising administering to the subject a cyclic polypeptide comprising a sequence of X-Cys-Y-Cys-Z, wherein X is an amino acid sequence of 0-30 amino acids, Y is an amino acid sequence of 2-20 amino acids, Z is an amino acid sequence of 0-30 amino acids, and at least two Cys are cysteine amino acids; and wherein the cyclic polypeptide detects an epitope of an antigen associated with the disease or disorder.

In one aspect, disclosed herein is a method of imaging a desired area in a subject, the method comprising administering to the subject a cyclic polypeptide comprising a sequence of X-Cys-Y-Cys-Z, wherein X is an amino acid sequence of 0-30 amino acids, Y is an amino acid sequence of 2-20 amino acids, Z is an amino acid sequence of 0-30 amino acids, and at least two Cys are cysteine amino acids; and wherein the cyclic polypeptide is operably linked to an imaging agent, wherein the imaging agent is targeted to the desired area and said desired area is imaged.

In some embodiments, the functional molecule comprises an antibody or fragments thereof, a diagnostic agent, or combinations thereof. In some embodiments, the antibody or fragments thereof comprise a single chain variable fragment (scFv). In some embodiments, the antibody or fragments thereof comprise a bispecific antibody. In some embodiments, the diagnostic agent comprises a radioisotope or a fluorescent molecule.

In some embodiments, the desired area comprises a tumor. In some embodiments, the imaging agent is useful in magnetic resonance. In some embodiments, the imaging agent comprises an iron oxide microparticle. In some embodiments, the imaging agent comprises a fluorescein molecule.

In some embodiments, the method comprises a cyclic polypeptide recognizing an epitope of the antigen.

In some embodiments, the method of any preceding aspect comprises 80%, 81%, 82%, 83%, 84%, 85%, 86%, 87%, 88%, 89%, 90%, 91%, 92%, 93%, 94%, 95%, 96%, 97%, 98%, 99% sequence identity to SEQ ID NO: 21. In some embodiments, the method of any preceding aspect comprises SEQ ID NO: 21

In one aspect, disclosed herein is a polypeptide comprising any one of SEQ ID NOS: 1-20.

### BRIEF DESCRIPTION OF FIGURES

The accompanying drawings, which are incorporated in and constitute a part of this specification, illustrate certain examples of the present disclosure and together with the description, serve to explain, without limitation, the principles of the disclosure. Like numbers represent the same elements throughout the figures.

FIG. 1 shows purification of cyclic linker mutant and control.

FIG. 2 shows differential scanning fluorimetry (DSF) of cyclic linker strains vs. wild type.

FIG. 3 shows that circular linkers are able to bind streptavidin, whereas linkers which are formed from glycine instead of cysteine don't circularize, and therefore aren't capable of binding streptavidin.

FIG. 4 shows streptavidin batch purification.

FIG. 5 shows streptavidin column purification.

FIG. 6 shows the structure of a typical antibody and a disulfide bridge.

FIG. 7 shows a single chain Fv fragment, a disulfide bridge, and a human IgG immunoglobulin.

FIG. 8 shows a general method for creating cysteine-free antibody fragments that are tested for thermal stability. General method: Express→Purify→Test thermal stability. Differential scanning fluorimetry (DSF): measures protein unfolding as the temperature increases using a fluorescent signal.

FIGS. 9A, 9B, and 9C show changing to alanine produces the most stable mutant in the light chain variable region (VL). FIG. 9A shows the mutation with A/A, FIG. 9B shows A/V, and FIG. 9C shows V/A.

FIGS. 10A and 10B shows that removing disulfide bonds in VH requires rigorous design. FIG. 10A shows various mutants, and FIG. 10B shows DSF of MR143 samples vs. wild type.

FIG. 11 shows thermal stability of cyclic linker variants.

FIG. 12 shows oligomeric states of cyclic linker variants. Elution at monomeric scFv indicating that the cysteine pair forms intramolecular cyclic cystine rather than intermolecular dimers.

FIG. 13 shows streptavidin binding cyclic linker test binding with protein L elution buffer

FIG. 14 shows streptavidin binding cyclic linker binding test in BME buffer (v/v %).

FIG. 15 shows streptavidin binding cyclic linker test binding with BME elution.

FIG. 16 shows streptavidin binding cyclic linker test binding with biotin elution.

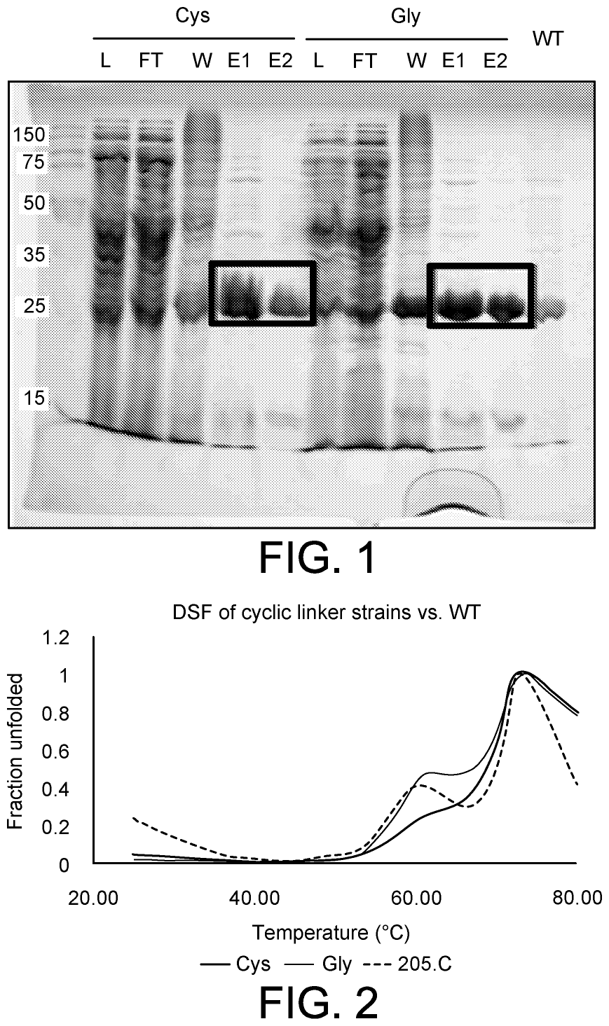

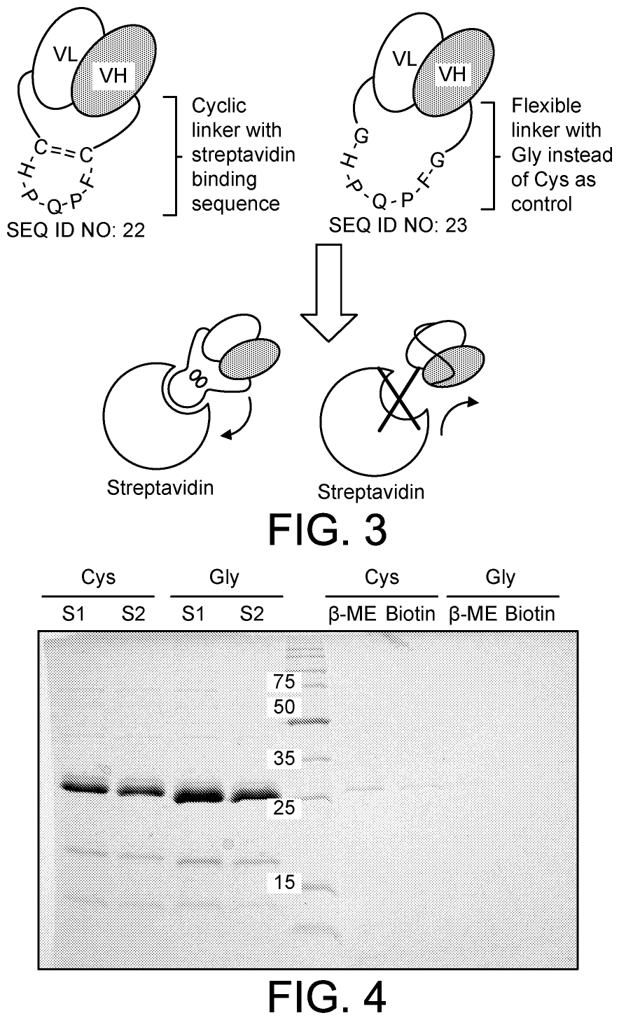

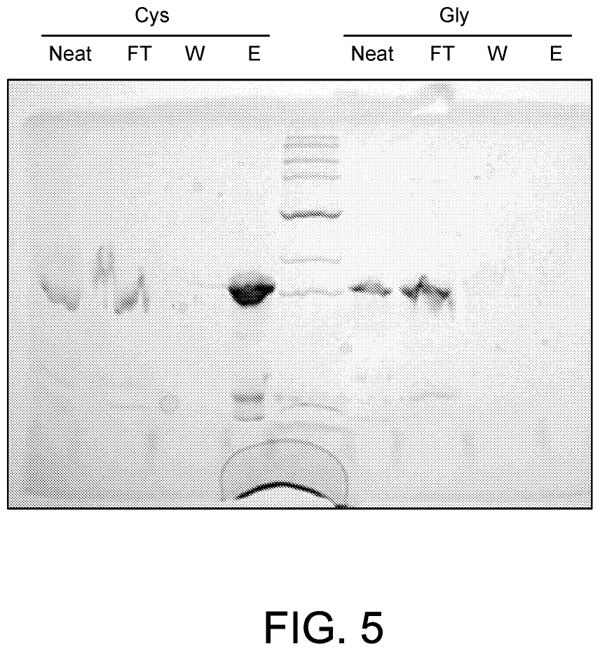

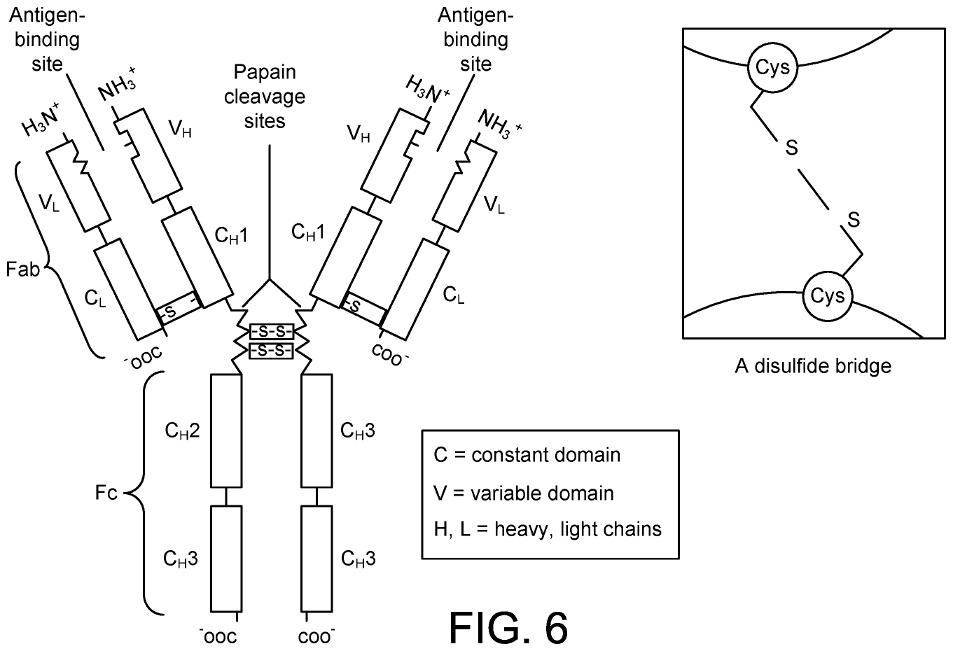

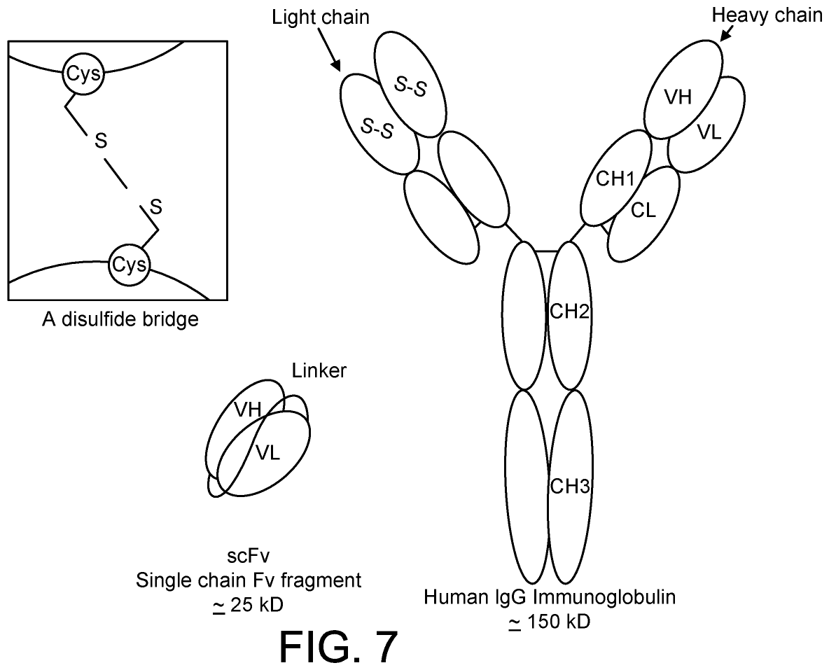

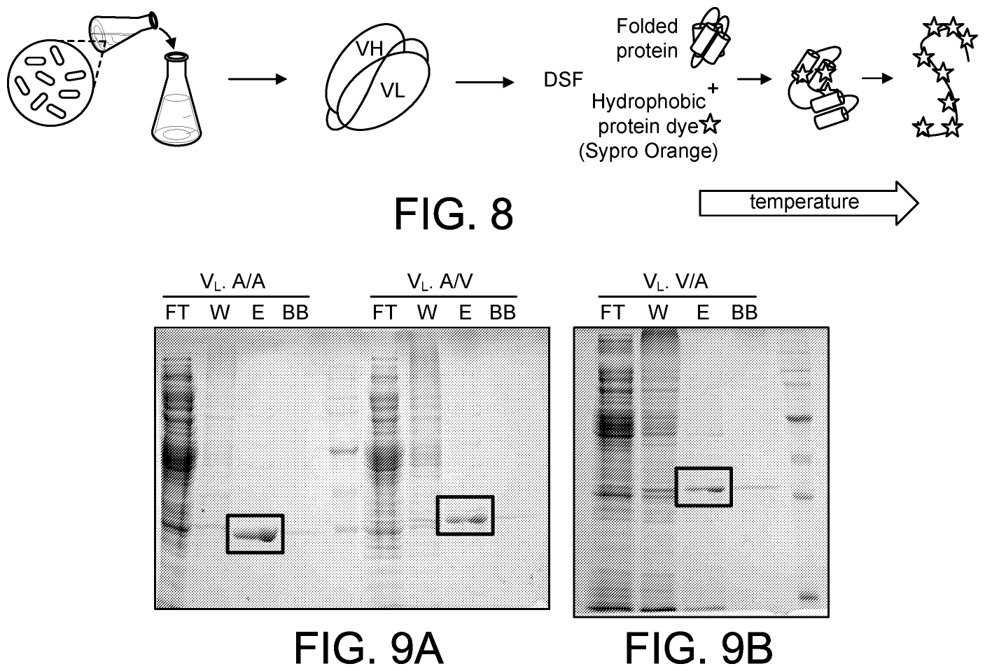

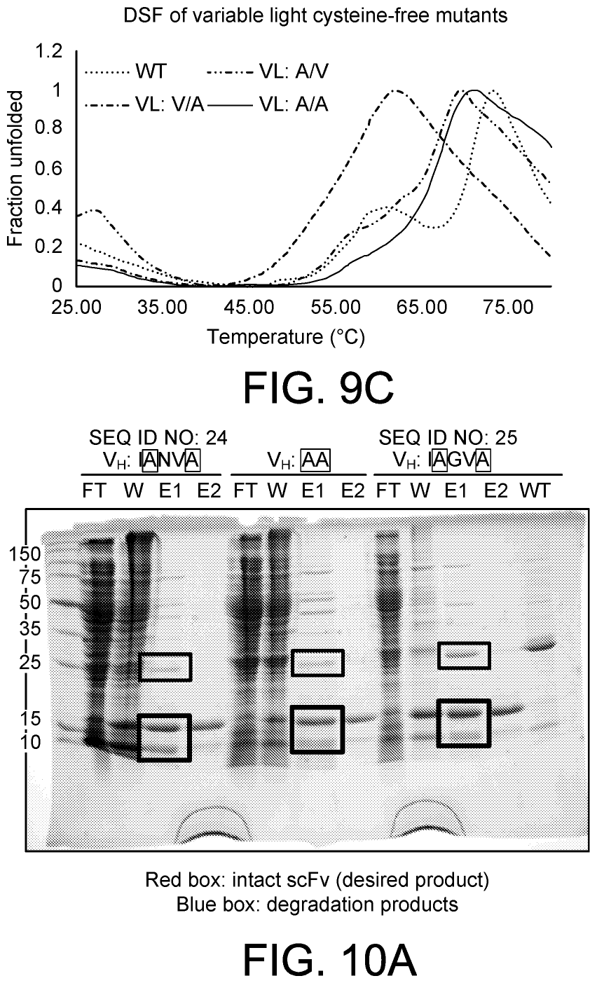

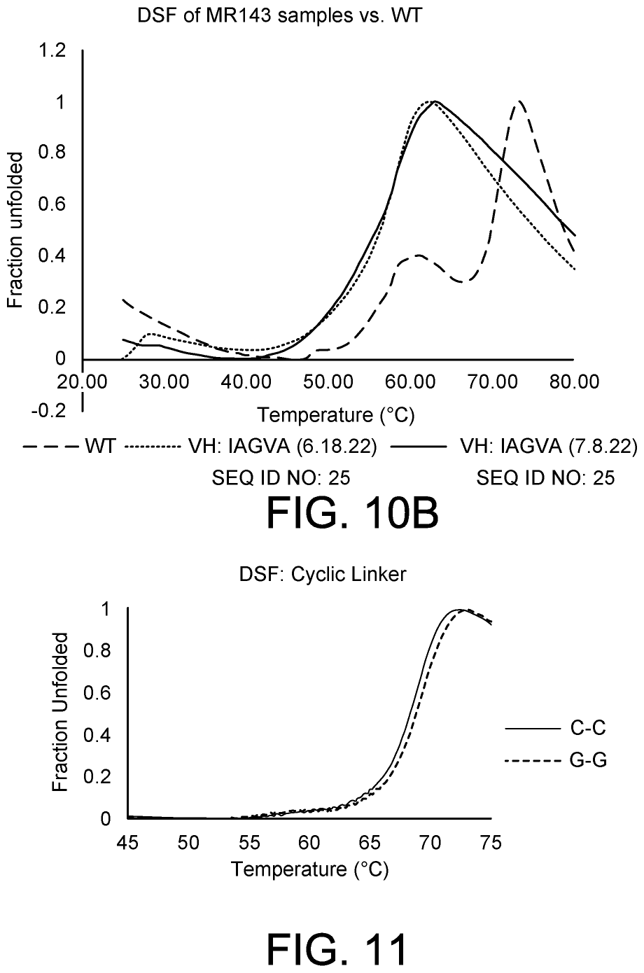

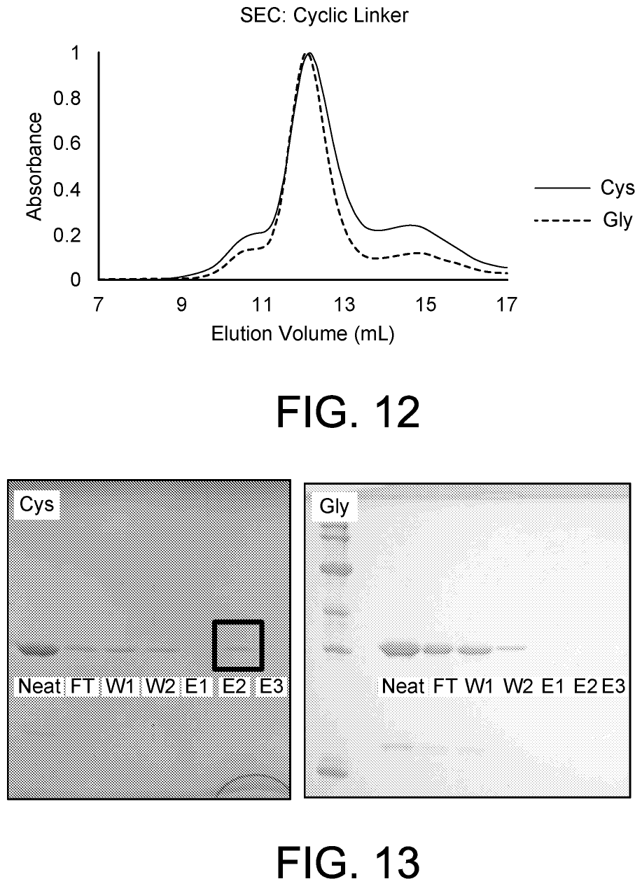

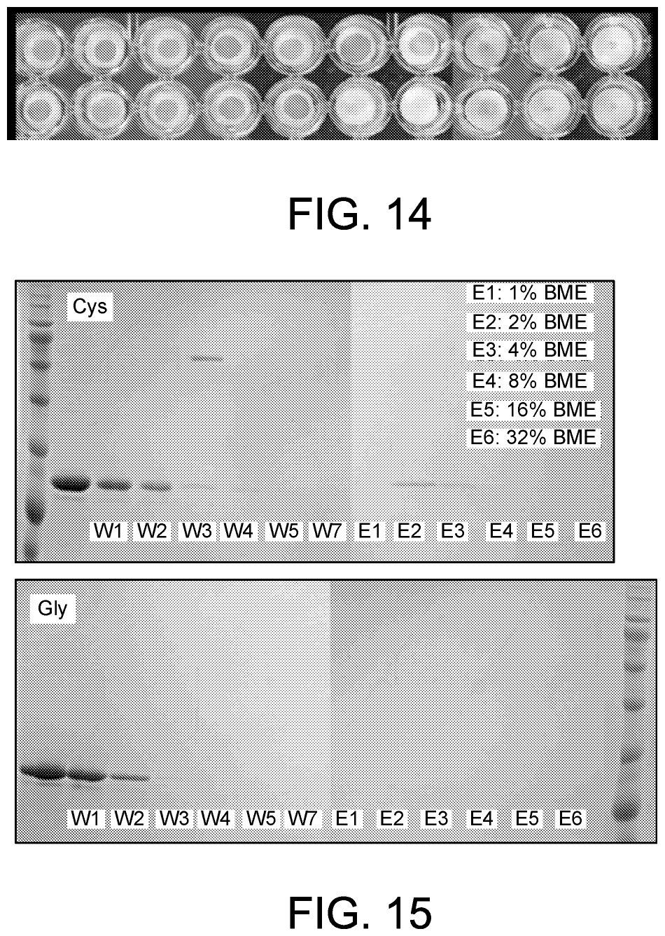

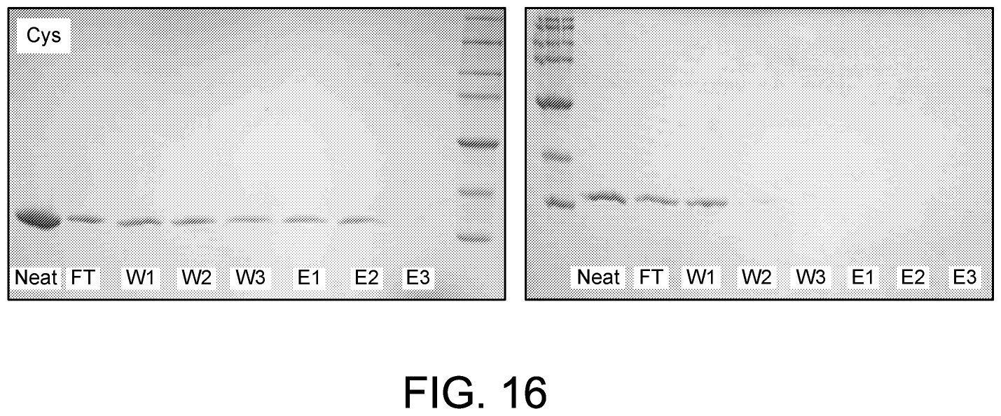

### DETAILED DESCRIPTION

The following description of the disclosure is provided as an enabling teaching of the disclosure in its best, currently known embodiment(s). To this end, those skilled in the relevant art will recognize and appreciate that many changes can be made to the various embodiments of the invention described herein, while still obtaining the beneficial results of the present disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Reference will now be made in detail to the embodiments of the invention, examples of which are illustrated in the drawings and the examples. This invention may, however, be embodied in many different forms and should not be construed as limited to the embodiments set forth herein.

### Terminology

In this specification and in the claims that follow, reference will be made to a number of terms, which shall be defined to have the following meanings.

Throughout the description and claims of this specification the word “comprise” and other forms of the word, such as “comprising” and “comprises,” means including but not limited to, and is not intended to exclude, for example, other additives, components, integers, or steps

As used in the description and the appended claims, the singular forms “a,” “an,” and “the” include plural referents unless the context clearly dictates otherwise. Thus, for example, reference to “a composition” includes mixtures of two or more such compositions, reference to “an agent” includes mixtures of two or more such agents, reference to “the component” includes mixtures of two or more such components, and the like.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. By “about” is meant within 5% of the value, e.g., within 4, 3, 2, or 1% of the value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, by a “subject” means an individual. Thus, the “subject” can include domesticated animals (e.g., cats, dogs, etc.), livestock (e.g., cattle, horses, pigs, sheep, goats, etc.), laboratory animals (e.g., mouse, rabbit, rat, guinea pig, etc.), and birds. “Subject” can also include a mammal, such as a primate or a human. Thus, the subject can be a human or veterinary patient. The term “patient” refers to a subject under the treatment of a clinician, e.g., physician.

The term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder, and supportive treatment, that is, treatment employed to supplement another specific therapy directed toward the improvement of the associated disease, pathological condition, or disorder.

The term “therapeutically effective” refers to the amount of the composition used is of sufficient quantity to ameliorate one or more causes or symptoms of a disease or disorder. Such amelioration only requires a reduction or alteration, not necessarily elimination.

The term “pharmaceutically acceptable” refers to those compounds, materials, compositions, and/or dosage forms which are, within the scope of sound medical judgment, suitable for use in contact with the tissues of human beings and animals without excessive toxicity, irritation, allergic response, or other problems or complications commensurate with a reasonable benefit/risk ratio.

The term “carrier” means a compound, composition, substance, or structure that, when in combination with a compound or composition, aids or facilitates preparation, storage, administration, delivery, effectiveness, selectivity, or any other feature of the compound or composition for its intended use or purpose. The term “carrier” also encompasses any excipient, diluent, filler, salt, buffer, stabilizer, solubilizer, lipid, stabilizer, or other material well known in the art for use in pharmaceutical formulations. The choice of a carrier for use in a composition will depend upon the intended route of administration for the composition. The preparation of pharmaceutically acceptable carriers and formulations containing these materials is described in, e.g., Remington's Pharmaceutical Sciences, 21st Edition, ed. University of the Sciences in

Philadelphia, Lippincott, Williams & Wilkins, Philadelphia, PA, 2005. Examples of physiologically acceptable carriers include saline, glycerol, DMSO, buffers such as phosphate buffers, citrate buffer, and buffers with other organic acids; antioxidants including ascorbic acid; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ (ICI, Inc.; Bridgewater, New Jersey), polyethylene glycol (PEG), and PLURONICS™ (BASF; Florham Park, NJ). For example, a carrier can be selected to minimize any degradation of the active ingredient and to minimize any adverse side effects in the subject.

As used herein in the context of molecules, the term “effector” refers to any molecule or combination of molecules whose activity it is desired to deliver/into and/or localize at a cell. Effectors include, but are not limited to labels, cytotoxins, enzymes, growth factors, transcription factors, drugs, etc.

As used herein in the context of cells of the immune system, the term “effector” refers to an immune system cell that can be induced to perform a specific function associated with an immune response to a stimulus. Exemplary effector cells include, but are not limited to natural killer (NK) cells and cytotoxic T cells (Tc cells).

As used herein, the term “expression vector” refers to a DNA sequence capable of directing expression of a particular nucleotide sequence in an appropriate host cell, comprising a promoter operatively linked to the nucleotide sequence of interest which is operatively linked to termination signals. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression.

As used herein, the term “hybridoma” refers to a cell or cell line that is produced in the laboratory from the fusion of an antibody-producing lymphocyte and a non-antibody-producing cancer cell, usually a myeloma or lymphoma cell. As would be known to those of one of ordinary skill in the art, a bybridoma can proliferate and produce a continuous supply of a specific monoclonal antibody. Methods for generating hybridomas are known in the art (see e.g., Harlow & Lane, 1988).

As used herein, the term “prodrug” refers to an analog and/or a precursor of a drug (e.g., a cytotoxic agent) that substantially lacks the biological activity of the drug (e.g., a cytotoxic activity) until subjected to an activation step. Activation steps can include enzymatic cleavage, chemical activation steps such as exposure to a reductant, and/or physical activation steps such as photolysis. In some embodiments, activation occurs in vivo within the body of a subject,

As used herein, the terms “antibody” and “antibodies” refer to proteins comprising one or more polypeptides substantially encoded by immunoglobulin genes or fragments of immunoglobulin genes. Immunoglobulin genes typically include the kappa (κ), lambda (λ), alpha (α), gamma (γ), delta (δ), epsilon(ϵ), and mu (μ) constant region genes, as well as myriad immunoglobulin variable region genes. Light chains are classified as either κ or λ. In mammals, heavy chains are classified as γ, μ, α, δ, or ϵ, which in turn define the immunoglobulin classes, IgG, IgM, IgA, IgD, and IgE, respectively. Other species have other light and heavy chain genes (e.g., certain avians produced what is referred to as IgY, which is an immunoglobulin type that hens deposit in the yolks of their eggs), which are similarly encompassed by the presently disclosed subject matter. In some embodiments, the term “antibody” refers to an antibody that binds specifically to an epitope that is present on a tumor antigen.

The term “antibody fragment” refers to any derivative of an antibody which is less than full-length. In exemplary embodiments, the antibody fragment retains at least a significant portion of the full-length antibody's specific binding ability. Examples of antibody fragments include, but are not limited to, Fab, Fab′, F(ab′), scFv, Fv, diabody, tribody, tetrabody, Fd fragments, or mixtures thereof. The antibody fragment may be produced by any means. For instance, the antibody fragment may be enzymatically or chemically produced by fragmentation of an intact antibody, it may be recombinantly produced from a gene encoding the partial antibody sequence, or it may be wholly or partially synthetically produced. The antibody fragment may optionally be a single chain antibody fragment. Alternatively, the fragment may comprise multiple chains which are linked together, for instance, by disulfide linkages. The fragment may also optionally be a multimolecular complex.

A typical immunoglobulin (antibody) structural unit is known to comprise a tetramer. Each tetramer is composed of two identical pairs of polypeptide chains, each pair having one “light” chain (average molecular weight of about 25 kilodalton (kDa)) and one “heavy” chain (average molecular weight of about 50-70 kDa). The two identical pairs of polypeptide chains are held together in dimeric form by disulfide bonds that are present within the heavy chain region. The N-terminus of each chain defines a variable region of about 100 to 110 or more amino acids primarily responsible for antigen recognition (sometimes referred to as the “paratope”). The terms variable light chain (VL) and variable heavy chain (VH) refer to these light and heavy chains, respectively

Antibodies typically exist as intact immunoglobulins or as a number of well-characterized fragments that can be produced by digestion with various peptidases For example, digestion of an antibody molecule with papain cleaves the antibody at a position N-terminal to the disulfide bonds. This produces three fragments: two identical “Fab” fragments, which have a light chain and the N-terminus of the heavy chain, and an “Fc” fragment that includes the C-terminus of the heavy chains held together by the disulfide bonds. Pepsin, on the other hand, digests an antibody C-terminal to the disulfide bond in the hinge region to produce a fragment known as the “F(ab)′2” fragment, which is a dimer of the Fab fragments joined by the disulfide bond. The F(ab)′2 fragment can be reduced under mild conditions to break the disulfide linkage in the hinge region, thereby converting the F(ab′)2 dimer into two “Fab” monomers. The Fab' monomer is essentially a Fab fragment with part of the hinge region (see e.g., Paul, 1993, for a more detailed description of other antibody fragments). With respect to these various fragments, Fab, F(ab′)2, and Fab′ fragments include at least one intact antigen binding domain (paratope), and thus are capable of binding to antigens.

While various antibody fragments are defined in terms of the digestion of an intact antibody, one of skill will appreciate that various of these fragments (including, but not limited to Fab′ fragments) can be synthesized de novo either chemically or by utilizing recombinant DNA methodology. Thus, the term “antibody” as used herein also includes antibody fragments produced by the modification of whole antibodies and/or synthesized de novo using recombinant DNA methodologies. In some embodiments, the term “antibody” comprises a fragment that has at least one antigen binding domain (paratope).

Antibodies can be polyclonal or monoclonal. As used herein, the term “polyclonal” refers to antibodies that are present together in a given collection of antibodies and that are derived from different antibody-producing cells (e.g., B cells). Exemplary polyclonal antibodies include, but are not limited to those antibodies that bind to a particular antigen and that are found in the blood of an animal after that animal has produced an immune response against the antigen. However, it is understood that a polyclonal preparation of antibodies can also be prepared artificially by mixing at least non-identical two antibodies. Thus, polyclonal antibodies typically include different antibodies that are directed against (i.e., bind to) the same and/or different epitopes (sometimes referred to as an “antigenic determinant” or just “determinant”) of any given antigen.

As used herein, the term “monoclonal” refers to a single antibody species and/or a substantially homogeneous population of a single antibody species. Stated another way, “monoclonal” refers to individual antibodies or populations of individual antibodies in which the antibodies are identical in specificity and affinity except for possible naturally occurring mutations that can be present in minor amounts. Typically, a monoclonal antibody (mAb or moAb) is generated by a single B cell or a progeny cell thereof (although the presently disclosed subject matter also encompasses “monoclonal” antibodies that are produced by molecular biological techniques as described herein). Monoclonal antibodies (mAbs or moAbs) are highly specific, typically being directed against a single antigenic site. Furthermore, in contrast to polyclonal antibody preparations, a given mAb is typically directed against a single epitope on the antigen.

In addition to their specificity, mAbs can be advantageous for some purposes in that they can be synthesized uncontaminated by other antibodies. The modifier “monoclonal” is not to be construed as requiring production of the antibody by any particular method, however. For example, in some embodiments, the mAbs of the presently disclosed subject matter are prepared using the hybridoma methodology first described by Kohler et al., 1975, and in some embodiments are made using recombinant DNA methods in prokaryotic or eukaryotic cells (see e.g., U.S. Pat. No. 4,816,567, the entire contents of which are incorporated herein by reference). mAbs can also be isolated from phage antibody libraries.

The antibodies, fragments, and derivatives of the presently disclosed subject matter can also include chimeric antibodies. As used herein in the context of antibodies, the term “chimeric”, and grammatical variants thereof, refers to antibody derivatives that have constant regions derived substantially or exclusively from antibody constant regions from one species and variable regions derived substantially or exclusively from the sequence of the variable region from another species.

The variable region allows an antibody to selectively recognize and specifically bind epitopes on antigens. That is, the VL domain and VH domain, or subsets of the complementarity determining regions (CDRs) within these variable domains, of an antibody combine to form the variable region that defines a three dimensional antigen binding site. This quaternary antibody structure forms the antigen binding site present at the end of each arm of the antibody. More specifically, the antigen binding site is defined by three CDRs on each of the VH and VL chains. In some instances (e.g., certain immunoglobulin molecules derived from camelid species or engineered based on camelid immunoglobulins), a complete immunoglobulin molecule can consist of heavy chains only with no light chains.

In naturally occurring antibodies, there are six CDRs present in each antigen binding domain that are short, non-contiguous sequences of amino acids that are specifically positioned to form the antigen binding domain as the antibody assumes its three dimensional configuration in an aqueous environment. The remainder of the amino acids in the antigen binding domains, referred to as “framework” regions, show less inter-molecular variability. The framework regions largely adopt a β-sheet conformation and the CDRs form loops that connect, and in some cases form part of, the β-sheet structure. Thus, framework regions act to form a scaffold that provides for positioning the CDRs in correct orientation by inter-chain, non-covalent interactions. The antigen binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable domain by one of ordinary skill in the art, since they have been precisely defined (see e.g., Chothia & Lesk, 1987; Kabat et al., 1991; Martin, 1996; Johnson & Wu, 2000).

A particular kind of chimeric antibody is a “humanized” antibody, in which the antibodies are produced by substituting the CDRs of, for example, a mouse antibody, for the CDRs of a human antibody (see e.g., PCT International Patent Application Publication No. WO 1992/22653). Thus, in some embodiments, a humanized antibody has constant regions and variable regions other than the CDRs that are derived substantially or exclusively from the corresponding regions of a human antibody, and CDRs that are derived substantially or exclusively from a mammal other than a human.

Fv fragments correspond to the variable fragments at the N-termini of immunoglobulin heavy and light chains. Fv fragments appear to have lower interaction energy of their two chains than Fab fragments. To stabilize the association of the VH and VL domains, they can be linked with peptides (see e.g., Bird et al., 1988; Huston et al., 1988), disulfide bridges (see e.g., Glockshuber et al., 1990), and/or “knob in hole” mutations (see e.g., Zhu et al., 1997). ScFv fragments can be produced by methods well known to those skilled in the art (see e.g., Whitlow et al., 1991; Huston et al., 1993).

A “single-chain variable fragment” (scFv) is a fusion protein of the variable regions of the heavy (VH) and light chains (VL) of immunoglobulins, connected with a short linker peptide. The linker can be rich in glycine for flexibility, as well as serine or threonine for solubility, and can either connect the N-terminus of the VH with the C-terminus of the VL, or vice versa. This protein retains the specificity of the original immunoglobulin, despite removal of the constant regions and the introduction of the linker. scFv can be produced in bacterial cells such as E. coli or in eukaryotic cells.

Reference is made herein to nucleic acid and nucleic acid sequences. The terms “nucleic acid” and “nucleic acid sequence” refer to a nucleotide, oligonucleotide, polynucleotide (which terms may be used interchangeably), or any fragment thereof. These phrases also refer to DNA or RNA of genomic or synthetic origin (which may be single-stranded or double-stranded and may represent the sense or the antisense strand).

Reference also is made herein to peptides, polypeptides, proteins, and compositions comprising peptides, polypeptides, and proteins. As used herein, a polypeptide and/or protein is defined as a polymer of amino acids, typically of length≥100 amino acids (Garrett & Grisham, Biochemistry, 2nd edition, 1999, Brooks/Cole, 110). A peptide is defined as a short polymer of amino acids, of a length typically of 20 or less amino acids, and more typically of a length of 12 or less amino acids (Garrett & Grisham, Biochemistry, 2nd edition, 1999, Brooks/Cole, 110).

As disclosed herein, exemplary peptides, polypeptides, proteins may comprise, consist essentially of, or consist of any reference amino acid sequence disclosed herein, or variants of the peptides, polypeptides, and proteins may comprise, consist essentially of, or consist of an amino acid sequence having at least about 80%, 90%, 95%, 96%, 97%, 98%, or 99% sequence identity to any amino acid sequence disclosed herein. Variant peptides, polypeptides, and proteins may include peptides, polypeptides, and proteins having one or more amino acid substitutions, deletions, additions and/or amino acid insertions relative to a reference peptide, polypeptide, or protein. Also disclosed are nucleic acid molecules that encode the disclosed peptides, polypeptides, and proteins (e.g., polynucleotides that encode any of the peptides, polypeptides, and proteins disclosed herein and variants thereof).

The term “amino acid,” includes but is not limited to amino acids contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. Typically, the amide linkages of the peptides are formed from an amino group of the backbone of one amino acid and a carboxyl group of the backbone of another amino acid.

The peptides, polypeptides, and proteins disclosed herein may be modified to include non-amino acid moieties. Modifications may include but are not limited to carboxylation (e.g., N-terminal carboxylation via addition of a di-carboxylic acid having 4-7 straight-chain or branched carbon atoms, such as glutaric acid, succinic acid, adipic acid, and 4,4-dimethylglutaric acid), amidation (e.g., C-terminal amidation via addition of an amide or substituted amide such as alkylamide or dialkylamide), PEGylation (e.g., N-terminal or C-terminal PEGylation via additional of polyethylene glycol), acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as farnesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e g., of thyroid hormones), and phosphorylation (e.g., the addition of a phosphate group, usually to serine, tyrosine, threonine, or histidine).

Variants comprising deletions relative to a reference amino acid sequence or nucleotide sequence are contemplated herein. A “deletion” refers to a change in the amino acid or nucleotide sequence that results in the absence of one or more amino acid residues or nucleotides relative to a reference sequence. A deletion removes at least 1, 2, 3, 4, 5, 10, 20, 50, 100, or 200 amino acids residues or nucleotides. A deletion may include an internal deletion or a terminal deletion (e.g., an N-terminal truncation or a C-terminal truncation or both of a reference polypeptide or a 5′-terminal or 3′-terminal truncation or both of a reference polynucleotide).

Variants comprising a fragment of a reference amino acid sequence or nucleotide sequence are contemplated herein. A “fragment” is a portion of an amino acid sequence or a nucleotide sequence which is identical in sequence to but shorter in length than the reference sequence. A fragment may comprise up to the entire length of the reference sequence, minus at least one nucleotide/amino acid residue For example, a fragment may comprise from S to 1000 contiguous nucleotides or contiguous amino acid residues of a reference polynucleotide or reference polypeptide, respectively. In some embodiments, a fragment may comprise at least 5, 10. 15, 20, 21, 22, 23, 24, 25, 26, 27, 28, 29, 30, 40, 50, 60, 70, 80, 90, 100, 150, 250, or 500 contiguous nucleotides or contiguous amino acid residues of a reference polynucleotide or reference polypeptide, respectively. Fragments may be preferentially selected from certain regions of a molecule, for example the N-terminal region and/or the C-terminal region of a polypeptide or the S′-terminal region and/or the 3′ terminal region of a polynucleotide. The term “at least a fragment” encompasses the full length polynucleotide or full length polypeptide.

Variants comprising insertions or additions relative to a reference sequence are contemplated herein. The words “insertion” and “addition” refer to changes in an amino acid or nucleotide sequence resulting in the addition of one or more amino acid residues or nucleotides An insertion or addition may refer to 1, 2, 3, 4, 5, 10, 20, 30, 40, 50, 60, 70, 80, 90, 100, 150, or 200 amino acid residues or nucleotides.

Fusion proteins and fusion polynucleotides are also contemplated herein. A “fusion protein” refers to a protein formed by the fusion of at least one peptide, polypeptide, protein or variant thereof as disclosed herein to at least one molecule of a heterologous peptide, polypeptide, protein or variant thereof. The heterologous protein(s) may be fused at the N-terminus, the C-terminus, or both termini. A fusion protein comprises at least a fragment or variant of the heterologous protein(s) that are fused with one another, preferably by genetic fusion (i.e., the fusion protein is generated by translation of a nucleic acid in which a polynucleotide encoding all or a portion of a first heterologous protein is joined in-frame with a polynucleotide encoding all or a portion of a second heterologous protein). The heterologous protein(s), once part of the fusion protein, may each be referred to herein as a “portion”, “region” or “moiety” of the fusion protein.

A fusion polynucleotide refers to the fusion of the nucleotide sequence of a first polynucleotide to the nucleotide sequence of a second heterologous polynucleotide (e.g., the 3′ end of a first polynucleotide to a 5′ end of the second polynucleotide). Where the first and second polynucleotides encode proteins, the fusion may be such that the encoded proteins are in-frame and results in a fusion protein. The first and second polynucleotide may be fused such that the first and second polynucleotide are operably linked (e.g., as a promoter and a gene expressed by the promoter as discussed below).

“Homology” refers to sequence similarity or, interchangeably, sequence identity, between two or more polypeptide sequences or polynucleotide sequences. Homology, sequence similarity, and percentage sequence identity may be determined using methods in the art and described herein

The phrases “percent identity” and “% identity,” as applied to polypeptide sequences, refer to the percentage of residue matches between at least two polypeptide sequences aligned using a standardized algorithm. Methods of polypeptide sequence alignment are well-known. Some alignment methods take into account conservative amino acid substitutions. Such conservative substitutions, explained in more detail above, generally preserve the charge and hydrophobicity at the site of substitution, thus preserving the structure (and therefore function) of the polypeptide. Percent identity for amino acid sequences may be determined as understood in the art. (See, e.g., U.S. Pat. No. 7.396,664, which is incorporated herein by reference in its entirety). A suite of commonly used and freely available sequence comparison algorithms is provided by the National Center for Biotechnology Information (NCBI) Basic Local Alignment Search Tool (BLAST)

(Altschul, S. F. et al (1990) J. Mol. Biol. 215:403 410), which is available from several sources, including the NCBI, Bethesda, Md., at its website. The BLAST software suite includes various sequence analysis programs including “blastp,” that is used to align a known amino acid sequence with other amino acids sequences from a variety of databases.

Percent identity may be measured over the length of an entire defined polypeptide sequence or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length may be used to describe a length over which percentage identity may be measured.

A “variant” of a particular polypeptide sequence may be defined as a polypeptide sequence having at least 50% sequence identity to the particular polypeptide sequence over a certain length of one of the polypeptide sequences using blastp with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”, FEMS Microbiol Lett. 174.247-250) In some embodiments a variant polypeptide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polypeptide. A variant polypeptide may have substantially the same functional activity as a reference polypeptide. For example, a variant polypeptide may exhibit or more biological activities associated with binding a ligand and/or binding DNA at a specific binding site.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences Percent identity for a nucleic acid sequence may be determined as understood in the art. (See, e.g., U.S. Pat. No. 7,396,664, which is incorporated herein by reference in its entirety). A suite of commonly used and freely available sequence comparison algorithms is provided by the National Center for Biotechnology Information (NCBI) Basic Local Alignment Search Tool (BLAST) (Altschul, S. F. et al. (1990) J. Mol. Biol. 215:403 410), which is available from several sources, including the NCBI, Bethesda, Md., at its website. The BLAST software suite includes various sequence analysis programs including “blastn,” that is used to align a known polynucleotide sequence with other polynucleotide sequences from a variety of databases. Also available is a tool called “BLAST 2 Sequences” that is used for direct pairwise comparison of two nucleotide sequences. “BLAST 2 Sequences” can be accessed and used interactively at the NCBI website. The “BLAST 2 Sequences” tool can be used for both blastn and blastp (discussed above).

Percent identity may be measured over the length of an entire defined polynucleotide sequence or may be measured over a shorter length, for example, over the length of a fragment taken from a larger, defined sequence, for instance, a fragment of at least 20, at least 30, at least 40, at least 50, at least 70, at least 100, or at least 200 contiguous nucleotides. Such lengths are exemplary only, and it is understood that any fragment length may be used to describe a length over which percentage identity may be measured.

A “full length” polynucleotide sequence is one containing at least a translation initiation codon (e.g., methionine) followed by an open reading frame and a translation termination codon. A “full length” polynucleotide sequence encodes a “full length” polypeptide sequence.

A “variant,” “mutant,” or “derivative” of a particular nucleic acid sequence may be defined as a nucleic acid sequence having at least 50% sequence identity to the particular nucleic acid sequence over a certain length of one of the nucleic acid sequences using blastn with the “BLAST 2 Sequences” tool available at the National Center for Biotechnology Information's website. (See Tatiana A. Tatusova, Thomas L. Madden (1999), “Blast 2 sequences—a new tool for comparing protein and nucleotide sequences”. FEMS Microbiol Lett. 174:247-250). In some embodiments a variant polynucleotide may show, for example, at least 60%, at least 70%, at least 80%, at least 90%, at least 91%, at least 92%, at least 93%, at least 94%, at least 95%, at least 96%, at least 97%, at least 98%, or at least 99% or greater sequence identity over a certain defined length relative to a reference polynucleotide.

Nucleic acid sequences that do not show a high degree of identity may nevertheless encode similar amino acid sequences due to the degeneracy of the genetic code. It is understood that changes in a nucleic acid sequence can be made using this degeneracy to produce multiple nucleic acid sequences that all encode substantially the same protein. “Operably linked” refers to the situation in which a first amino acid sequence is placed in a functional relationship with a second amino acid sequence or any other molecule. For instance, a peptide or polypeptide molecule is operably or connected to another molecule, whether the molecule comprises therapeutic, diagnostic, or imaging, or is non-functional. The other molecule may be connected to the carboxyl terminal, the amino terminal, or both the carboxyl and the amino termini of the first amino acid sequence. In some embodiments, the first amino acid sequence and the second amino acid sequence are operably linked to generate a fusion peptide or polypeptide. In some embodiments, a first nucleic acid sequence can be operably linked to a second nucleic acid sequence, wherein both sequences together encode a fusion peptide or polypeptide.

A “recombinant nucleic acid” is a sequence that is not naturally occurring or has a sequence that is made by an artificial combination of two or more otherwise separated segments of sequence. This artificial combination is often accomplished by chemical synthesis or, more commonly, by the artificial manipulation of isolated segments of nucleic acids, e.g., by genetic engineering techniques such as those described in Sambrook, J. et al. (1989) Molecular Cloning: A Laboratory Manual, 2nd ed., vol. 13, Cold Spring Harbor Press, Plainview N.Y. The term recombinant includes nucleic acids that have been altered solely by addition, substitution, or deletion of a portion of the nucleic acid. Frequently, a recombinant nucleic acid may include a nucleic acid sequence operably linked to a promoter sequence. Such a recombinant nucleic acid may be part of a vector that is used, for example, to transform a cell.

“Transformation” describes a process by which exogenous DNA is introduced into a recipient cell. Transformation may occur under natural or artificial conditions according to various methods well known in the art, and may rely on any known method for the insertion of foreign nucleic acid sequences into a prokaryotic or eukaryotic host cell. The method for transformation is selected based on the type of host cell being transformed and may include, but is not limited to, bacteriophage or viral infection, electroporation, heat shock, lipofection, and particle bombardment. The term “transformed cells” includes stably transformed cells in which the inserted DNA is capable of replication either as an autonomously replicating plasmid or as part of the host chromosome, as well as transiently transformed cells which express the inserted DNA or RNA for limited periods of time.

“Substantially isolated or purified” nucleic acid or amino acid sequences are contemplated herein. The term “substantially isolated or purified” refers to nucleic acid or amino acid sequences that are removed from their natural environment, and are at least 60% free, preferably at least 75% free, and more preferably at least 90% free, even more preferably at least 95% free from other components with which they are naturally associated.

Reference will now be made in detail to specific aspects of the disclosed materials, compounds, compositions, articles, and methods, examples of which are illustrated in the accompanying Examples and Figures.

### Cyclic Polypeptides Compositions

In nature, the formation of disulfide bonds between cysteine residues occurs during the folding of many proteins that enter the secretory pathway. As the polypeptide chain collapses, cysteines brought into proximity can form covalent linkages during a process catalyzed by members of the protein disulfide isomerase family (Bulleid 2012). A disulfide bridge is created when a sulfur atom from one cysteine forms a single covalent bond with another sulfur atom from a second cysteine residue located in a different part of the protein. These bridges help to stabilize proteins, particularly those secreted from cells.

In some examples, cysteine residues, which form a disulfide bridge, can be used to circularize the polypeptide. However, strategies to cyclize a peptide are known to those of skill in the art. For example, bismuth²⁺ can be used to make bicyclic compounds from peptides with three cysteines. Another way to cyclize is to use a dibromide small molecule with two cysteines. An acidic amino acid, such as Glu or Asp can be used with, for example, EDC, and it can react with a Lys. Synthetic, or unnaturally occurring amino acids can also be used. Examples include, but are not limited to, click chemistry (azide plus alkyne) or olefin metathesis. Deyle et al. (2017) and Gang (2018), both incorporated by reference, disclose how to make cyclic peptides which can be used with the invention disclosed herein

In one aspect, disclosed herein is a synthetic cyclic polypeptide comprising an amino acid sequence of X-Cys-Y-Cys-Z, wherein X is an amino acid sequence of 0-30 amino acids, Y is an amino acid sequence of 2-20 amino acids, Z is an amino acid sequence of 0-30 amino acids, and Cys is a cysteine residue; wherein the cysteine residues can form a disulfide bridge. In one aspect, disclosed herein is a peptide-antibody complex comprising a cyclic polypeptide operably linked to an antibody or fragments thereof, wherein the cyclic polypeptide comprises an amino acid sequence of X-Cys-Y-Cys-Z, wherein X is an amino acid sequence of 0-30 amino acids, Y is an amino acid sequence of 2-20 amino acids, Z is an amino acid sequence of 0-30 amino acids, and at least two Cys are cysteine amino acids; and wherein the antibody or fragments thereof comprise a single chain variable fragment (scFv).

In some embodiments, X is an amino acid sequence of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 amino acids. In some embodiments, Y is an amino acid sequence of 2, 3, 4, 5, 6, 7, 8, 9, 10, 11, 12, 13, 14, 15, 16, 17, 18, 19, 20 amino acids. In some embodiments, Z is an amino acid sequence of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 amino acids.

The cyclic polypeptide can be associated with a functional molecule at its C terminus, N terminus, or both the C and N terminus. Herein, “functional molecule” means a molecule that is desired for delivery either in vivo or in vitro. For in vitro purposes, the functional molecule can be used in diagnostics or imaging, or to identify a target molecule or effector. The cyclic peptides can be used in high throughput assays. When used in vivo, the functional molecule can be used for therapeutic or diagnostic uses. For example, the functional molecule can be delivered to a specific target to provide therapy. The functional molecule can be a polypeptide, such as an antibody or other therapeutic or diagnostic polypeptide, it can be a nucleic acid, such as one that would be used in RNA therapy, it can be a small molecule, a dye, a radioisotope, or a fluorescent molecule, for example.

The cyclic polypeptide can have a functional molecule on either the N terminus, the C terminus, or both. When the cyclic polypeptide bas a functional molecule on both ends, it can function as a linker. The cyclic peptide itself can serve a function. Cyclic peptides have several beneficial properties unlike their linear counterparts. Firstly, the rigidity of cyclic peptides increases binding affinity and selectivity toward target molecules, due to a decreased entropy term in the Gibbs free energy equation (Edman, 1959; Horton et al., 2002). Secondly, cyclic peptides are resistant to hydrolysis by exopeptidase as they lack amino and carboxyl ends. Cyclic peptides can even be resistant to endopeptidases if the rigid ring structure prevents endopeptidase from accessing the cleavage site. Thirdly, some cyclic peptides show better membrane permeability and can cross the cell membrane (Kwon and Kodadek, 2007, Choi 2020). Furthermore, the cyclic polypeptide can be used as a means of capture, as it can interact with another molecule which would allow for its capture. As seen in Example 1, some cyclic polypeptides have been shown to interact with streptavidin.

In some embodiments, the at least two Cys form a disulfide bridge. In some embodiments, the scFv comprises a heavy chain and a light chain. In some embodiments, the antibody or fragments thereof comprises a bispecific antibody. In some embodiments, the antibody or fragments thereof are operably linked to a C terminus, a N terminus, or both C and N termini of the cyclic polypeptide. Herein, “bispecific antibody” means an antibody or antibodies with two binding sites directed at two different antigens or two different epitopes on the same antigen. The cyclic polypeptide disclosed herein can be used as a functional binding site as well, due to the cyclic nature of the polypeptide. For example, the cyclic polypeptide can recognize an epitope Examples of bispecific antibodies can be found in Labrijn et al. (2019), herein incorporated by reference in its entirety for its teaching concerning bispecific antibodies. Disclosed herein is a cyclic polypeptide comprising GGGGSGGGGS-CHPQPFC-GGGGSGGGGS (SEQ ID NO: 21).

Bi-specific antibodies serve as promising therapeutic agents due to their ability to simultaneously bind two different antigens. Generating a bispecific antibody wherein one of the antigen recognition sites comprises a cyclic peptide enhances the chemical stability and antigen binding functions of the antibody. The ability of the cyclic peptide to recognize epitopes and/or enhance epitope binding of the antibody relies on the optimal mutations to the amino acid sequences of the cyclic polypeptide. Examples of peptide-antibody complexes can be found in Doppalapudi et al (2010), herein incorporated by reference in its entirety for its teachings of fusing peptides with an antibody scaffold using a linker. Herein, the present disclosure provides a bispecific antibody complex comprising an antibody or fragments thereof, and one cyclic peptide that can become a linker due to disulfide bridge formation between at least two cysteine amino acids on the one cyclic peptide. In some embodiments, the peptide-antibody complex comprises a cyclic polypeptide that recognizes a first epitope and a scFv that recognizes a second epitope. In some embodiments, the first epitope and the second epitope are located on the same antigen. In some embodiments, the first epitope and the second epitope are located on two different antigens. It is noted that the peptide-antibody complex disclosed herein comprises a bispecific antibody, wherein at least one of the binding sites of the bispecific antibody is located on the cyclic polypeptide segment(s) of the complex.

In some embodiments, the cyclic polypeptide becomes a linker when the antibody or fragments thereof are on both the N and C terminus of the cyclic polypeptide. In some embodiments, the cyclic polypeptide becomes a linker when the heavy chain of the scFv is on one side of the linker and the light chain of the scFv is on another side of the linker.

In some embodiments, the disulfide bridge forms a loop or a circle within the complex. In some embodiments, the loop, or the circle form spontaneously In some embodiments, the cyclic polypeptide can function as a linker, such as in a diabody, triabody, tetrabody, etc. In some embodiments, the scFv comprises a diabody or a tetrabody.

A non-limiting example of a peptide-antibody complex can be found at Example 1 of the present disclosure, wherein a streptavidin binding cyclic peptide, C-HPQGPP-C (SEQ ID NO: 1), was incorporated into 3E8, an scFv with affinity for siayl-Tn antigen. It should be noted that bovine serum albumin (BSM) was included in this example as an antigen source rich in siayl-Tn. Other non-limiting examples of the peptides and antibodies that can be incorporated in the complexes of any preceding aspect can be found in Long et al. (2018), herein incorporated by reference in its entirety for its teaching concerning fusing scFv antibodies, such as 3E8, to peptide linkers, and can be found in Giebel et al. (1995), herein incorporated by reference in its entirety for teaching concerning cyclic peptides targeting streptavidin.

In some embodiments, the cyclic polypeptide comprises at least 80% sequence identity to SEQ ID NO: 21. In some embodiments, the cyclic polypeptide comprises 80, 81, 82, 83, 84, 85, 86, 87, 88, 89, 90, 91, 92, 93, 94, 95, 96, 97, 98, or 99% sequence identity to SEQ ID NO: 21. In some embodiments, the cyclic polypeptide comprises SEQ ID NO: 21.

In one aspect, disclosed herein is a synthetic nucleic acid encoding the cyclic polypeptide of any preceding aspect. This can allow for its expression in a cell. The cyclic polypeptide can be in a vector, for example. Heterologous expression of the cyclic peptide, optionally along with a functional molecule, can be accomplished by one of skill in the art.

In one aspect, disclosed herein is a vector comprising the nucleic acid of any preceding aspect. Herein, the vector can be any expression vector known in the art to allow for stable and/or transient expression of the cyclic polypeptide. In some embodiments, the expression vector comprises a plasmid or a virus or viral vector. A plasmid or a viral vector can be capable of extrachromosomal replication or, optionally, can integrate into the host genome. As used herein, the term “integrated” used in reference to an expression vector (e.g., a plasmid or viral vector) means the expression vector, or a portion thereof, is incorporated (physically inserted or ligated) into the chromosomal DNA of a host cell. As used herein, a “viral vector” refers to a virus-like particle containing genetic material which can be introduced into a eukaryotic cell without causing substantial pathogenic effects to the eukaryotic cell. A wide range of viruses or viral vectors can be used for transduction but should be compatible with the cell type the virus or viral vector are transduced into (e.g., low toxicity, capability to enter cells). Suitable viruses and viral vectors include adenovirus, lentivirus, retrovirus, among others. In some embodiments, the expression vector encoding a cyclic polypeptide is a naked DNA or is comprised in a nanoparticle (e.g., liposomal vesicle, porous silicon nanoparticle, gold-DNA conjugate particle, polyethyleneimine polymer particle, cationic peptides, etc.)

In one aspect, disclosed herein is a cell comprising the vector of any preceding aspect. It should be understood that the cell comprising the vector can be any cell known in the art to stably or transiently express extrachromosomal genetic material. The terms “cell,” “cell line” and “cell culture” include progeny. It is also understood that all progenies may not be precisely identical in DNA content, due to deliberate or inadvertent mutations. Variant progeny that have the same function or biological property, as screened for in the originally transformed cell, are included. The cells used in the present disclosure generally are derived from prokaryotic (i.e.: bacterial) or eukaryotic (i.e.: mammalian) hosts.

In one aspect, disclosed herein is a polypeptide comprising any one of SEQ ID NOS. 1-20, or variants thereof. In some embodiments, the polypeptide comprises SEQ ID NO: 1, or variants thereof. In some embodiments, the polypeptide comprises SEQ ID NO: 2, or variants thereof. In some embodiments, the polypeptide comprises SEQ ID NO: 3, or variants thereof. In some embodiments, the polypeptide comprises SEQ ID NO: 4, or variants thereof. In some embodiments, the polypeptide comprises SEQ ID NO: 5, or variants thereof. In some embodiments, the polypeptide comprises SEQ ID NO: 6, or variants thereof. In some embodiments, the polypeptide comprises SEQ ID NO: 7, or variants thereof. In some embodiments, the polypeptide comprises SEQ ID NO: 8, or variants thereof. In some embodiments, the polypeptide comprises SEQ ID NO: 9, or variants thereof. In some embodiments, the polypeptide comprises SEQ ID NO 10, or variants thereof. In some embodiments, the polypeptide comprises SEQ ID NO: 11, or variants thereof. In some embodiments, the polypeptide comprises SEQ ID NO 12, or variants thereof. In some embodiments, the polypeptide comprises SEQ ID NO 13, or variants thereof. In some embodiments, the polypeptide comprises SEQ ID NO 14, or variants thereof. In some embodiments, the polypeptide comprises SEQ ID NO 15, or variants thereof In some embodiments, the polypeptide comprises SEQ ID NO: 16, or variants thereof. In some embodiments, the polypeptide comprises SEQ ID NO: 17, or variants thereof. In some embodiments, the polypeptide comprises SEQ ID NO: 18, or variants thereof. In some embodiments, the polypeptide comprises SEQ ID NO: 19, or variants thereof. In some embodiments, the polypeptide comprises SEQ ID NO: 20, or variants thereof.

The functional polypeptides, including the cyclic polypeptides of the present disclosure, can be operably linked to the functional molecule of any preceding aspect.

The functional polypeptides, including the cyclic polypeptides of the present disclosure, can be engineered so that they do not have cysteine residues (such as, for example the polypeptide sequence shown in SEQ ID NO: 23). This can aid in preventing undesired interactions.

### Methods of diagnosing and/or imaging

In one aspect, disclosed herein is a method of diagnosing a subject with a disease or disorder, the method comprising administering to the subject a cyclic polypeptide comprising a sequence of X-Cys-Y-Cys-Z, wherein X is an amino acid sequence of 0-30 amino acids, Y is an amino acid sequence of 2-20 amino acids, Z is an amino acid sequence of 0-30 amino acids, and at least two Cys are cysteine amino acids; and wherein the cyclic polypeptide detects an epitope of an antigen associated with the disease or disorder.

The method can be used to diagnose one or more diseases or disorders including, but not limited to cancer (such as, for example acoustic neuroma, adenocarcinoma, adrenal gland cancer, anal cancer, angiosarcoma (e.g., lymphangiosarcoma, lymphangioendotheliosarcoma, hemangiosarcoma), appendix cancer, benign monoclonal gammopathy, biliary cancer (e.g., cholangiocarcinoma), bladder cancer, breast cancer (e.g., adenocarcinoma of the breast, papillary carcinoma of the breast, mammary cancer, medullary carcinoma of the breast), brain cancer (e.g., meningioma; glioma, e.g., astrocytoma, oligodendroglioma; medulloblastoma), bronchus cancer, carcinoid tumor, cervical cancer (e.g., cervical adenocarcinoma), choriocarcinoma, chordoma, craniopharyngioma, colorectal cancer (e.g., colon cancer, rectal cancer, colorectal adenocarcinoma), epithelial carcinoma, ependymoma, endotheliosarcoma (e.g., Kaposi's sarcoma, multiple idiopathic hemorrhagic sarcoma), endometrial cancer (e.g., uterine cancer, uterine sarcoma), esophageal cancer (e.g., adenocarcinoma of the esophagus, Barrett's adenocarinoma), Ewing's sarcoma, eye cancer (e.g., intraocular melanoma, retinoblastoma), familiar hypereosinophilia, gall bladder cancer, gastric cancer (e.g., stomach adenocarcinoma), gastrointestinal stromal tumor (GIST), head and neck cancer (e.g., head and neck squamous cell carcinoma, oral cancer (e.g., oral squamous cell carcinoma (OSCC), throat cancer (e.g., laryngeal cancer, pharyngeal cancer, nasopharyngeal cancer, oropharyngeal cancer)), hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma (DLBCL)), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., “Waldenstrom's macroglobulinemia”), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungiodes, Sezary syndrome), angioimmunoblastic T-cell lymphoma, extranodal natural killer T-cell lymphoma, enteropathy type T-cell lymphoma, subcutaneous panniculitis-like T-cell lymphoma, anaplastic large cell lymphoma); a mixture of one or more leukemia/lymphoma as described above; and multiple myeloma (MM)), heavy chain disease (e.g., alpha chain disease, gamma chain disease, mu chain disease), hemangioblastoma, inflammatory myofibroblastic tumors, immunocytic amyloidosis, kidney cancer (e.g., nephroblastoma a.k.a. Wilms' tumor, renal cell carcinoma), liver cancer (e.g., hepatocellular cancer (HCC), malignant hepatoma), lung cancer (e.g., bronchogenic carcinoma, small cell lung cancer (SCLC), non-small cell lung cancer (NSCLC), adenocarcinoma of the lung), leiomyosarcoma (LMS), mastocytosis (e.g., systemic mastocytosis), myelodysplastic syndrome (MDS), mesothelioma, myeloproliferative disorder (MPD) (e.g., polycythemia Vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)), neuroblastoma, neurofibroma (e.g., neurofibromatosis (NF) type 1 or type 2, schwannomatosis), neuroendocrine cancer (e.g., gastroenteropancreatic neuroendoctrine tumor (GEP-NET), carcinoid tumor), osteosarcoma, ovarian cancer (e.g., cystadenocarcinoma, ovarian embryonal carcinoma, ovarian adenocarcinoma), papillary adenocarcinoma, pancreatic cancer (e.g., pancreatic adenocarcinoma, intraductal papillary mucinous neoplasm (IPMN), Islet cell tumors), penile cancer (e.g., Paget's disease of the penis and scrotum), pinealoma, primitive neuroectodermal tumor (PNT), prostate cancer (e.g., prostate adenocarcinoma), rectal cancer, rhabdomyosarcoma, salivary gland cancer, skin cancer (e.g., squamous cell carcinoma (SCC), keratoacanthoma (KA), melanoma, basal cell carcinoma (BCC)), small bowel cancer (e.g., appendix cancer), soft tissue sarcoma (e.g., malignant fibrous histiocytoma (MFH), liposarcoma, malignant peripheral nerve sheath tumor (MPNST), chondrosarcoma, fibrosarcoma, myxosarcoma), sebaceous gland carcinoma, sweat gland carcinoma, synovioma, testicular cancer (e.g., seminoma, testicular embryonal carcinoma), thyroid cancer (e.g., papillary carcinoma of the thyroid, papillary thyroid carcinoma (PTC), medullary thyroid cancer), urethral cancer, vaginal cancer and vulvar cancer (e.g., Paget's disease of the vulva)), neurodegenerative diseases (such as, for example Alzheimer's disease, ataxia, Huntington's disease, Parkinson's disease, amyotrophic lateral sclerosis (ALS), Friedreich ataxia, Lewy body disease, spinal muscular atrophy, Alpers' disease, Batten disease, Cerebro-oculo-facio-skeletal syndrome, Leigh syndrome, Prion diseases, monomelic amyotrophy, multiple system atrophy, striatonigral degeneration, motor neuron disease, multiple sclerosis (MS), Creutzfeldt-Jakob disease, Parkinsonism, spinocerebellar ataxia, dementia, and other related diseases), infectious diseases (such as, for example common cold, influenza (including, but not limited to human, bovine, avian, porcine, and simian strains of influenza), measles, acquired immune deficiency syndrome/human immunodeficiency virus (AIDS/HIV), anthrax, botulism, cholera, campylobacter infections, chickenpox, chlamydia infections, cryptosporidosis, dengue fever, diphtheria, hemorrhagic fevers, Escherichia coli (E. coli) infections, ehrlichiosis, gonorrhea, hand-foot-mouth disease, hepatitis A, hepatitis B, hepatitis C, legionellosis, leprosy, leptospirosis, listeriosis, malaria, meningitis, meningococcal disease, mumps, pertussis, polio, pneumococcal disease, paralytic shellfish poisoning, rabies, rocky mountain spotted fever, rubella, salmonella, shigellosis, small pox, syphilis, tetanus, trichinosis (trichinellosis), tuberculosis (TB), typhoid fever, typhus, west Nile virus, yellow fever, yersiniosis, zika, and diseases caused by other infectious pathogens), cardiovascular diseases (such as, for example coronary artery disease, high/low blood pressure, cardiac arrest/heart failure, congestive heart failure, congenital heart defects/diseases (including, but not limited to atrial septal defects, atrioventricular septal defects, coarctation of the aorta, double-outlet right ventricle, d-transposition of the great arteries, Ebstein anomaly, hypoplastic left heart syndrome, and interrupted aortic arch), arrhythmia, peripheral artery disease, stroke, cerebrovascular disease, renal artery stenosis, aortic aneurysm, cardiomyopathies, hypertensive heart disease, pulmonary heart disease, cardiac dysrhythmias, endocarditis, inflammatory cardiomegaly, myocarditis, eosinophilic myocarditis, valvular heart diseases, rheumatic heart diseases, alcoholic cardiomyopathy, systemic carnitine deficiency, malonyl carboxylase deficiency, malonic aciduria, carnitine-acylcarnitine translocase deficiency, carnitine palmitoyltransferase II deficiency, deficiencies to mitochondrial beta-oxidation (including, but not limited to medium-chain acyl-coenzyme A (coA) dehydrogenase (MCAD) deficiency, short-chain acyl-coA dehydrogenase (SCAD) deficiency, very-long-chain acyl-coA dehydrogenase (VLCAD) deficiency, and long-chain 3-hydroxyacyl-coA dehydrogenase (LCHAD) deficiency), deficiencies to the mitochondrial electron respiratory chain (including, but not limited to Kearns-Sayre syndrome, MELAS syndrome, MERRF syndrome, Barth syndrome, Leigh's syndrome. Pearson syndrome, respiratory chain complex I deficiency, and Complex III deficiency), Glycogen storage disease type II (Pompe disease), Glycogen storage disease type III, Niemann-Pick disease, Gaucher disease, I-cell disease, mucopolysaccharidosis type I (Hurler syndrome), mucopolysaccharidosis type II (Hunter syndrome), mucopolysaccharidosis type III (Harris-Sanfilippo syndrome), mucopolysaccharidosis type IV (Morquio syndrome), mucopolysaccharidosis type VI (Maroteaux-Lamy syndrome), GM1 gangliosidosis, galactosialidosis, carbohydrate deficient glycoprotein syndromes, Sandhoff's disease, congenital heart defects and other related cardiovascular diseases), respiratory diseases (such as, for example asthma, chronic obstructive pulmonary disease (COPD), pulmonary fibrosis, pneumonia, bronchitis (chronic or acute bronchitis), emphysema, cystic fibrosis/bronchiectasis, pleural effusion, acute chest syndrome, acute respiratory distress syndrome, asbestosis, aspergillosis, severe acute respiratory syndrome (including, but not limited to SARS-COV-1 and SARS-COV-2), respiratory syncytial virus (RSV), middle eastern respiratory syndrome (MERS), mesothelioma, pneumothorax, pulmonary arterial hypertension, pulmonary hypertension, pulmonary embolism, sarcoidosis, sleep apnea, and other respiratory diseases), gastrointestinal diseases (such as, for example heartburn, irritable bowel syndrome, lactose intolerance, gallstones, cholecystitis, cholangitis, anal fissure, hemorrhoids, proctitis, colon polyps, infective colitis, ulcerative colitis, ischemic colitis, Crohn's disease, radiation colitis, celiac disease, diarrhea (chronic or acute), constipation (chronic or acute), diverticulosis, diverticulitis, acid reflux (gastroesophageal reflux (GER) or gastroesophageal reflux disease (GERD)), Hirschsprung disease, abdominal adhesions, achalasia, acute hepatic porphyria (AHP), anal fistulas, bowel incontinence, centrally mediated abdominal pain syndrome (CAPS), clostridioides difficile infection, cyclic vomiting syndrome (CVS), dyspepsia, eosinophilic gastroenteritis, globus, inflammatory bowel disease, malabsorption, scleroderma, volvulus, and other gastrointestinal diseases), and metabolic diseases (such as, for example diabetes mellitus Type I, diabetes mellitus Type II, familial hypercholesterolemia, Gaucher disease, Hunter syndrome, Krabbe syndrome, metachromatic leukodystrophy, Niemann-Pick syndrome, phenylketonuria (PKU), Tay-Sachs disease, Wilson's disease, hemachromatosis, mitochondrial disorders or diseases (including, but not limited to Alpers Disease; Barth syndrome; beta.-oxidation defects: carnitine-acyl-carnitine deficiency; carnitine deficiency; coenzyme Q10 deficiency; Complex I deficiency; Complex II deficiency; Complex III deficiency; Complex IV deficiency: Complex V deficiency: cytochrome c oxidase (COX) deficiency, LHON Leber Hereditary Optic Neuropathy; MM Mitochondrial Myopathy: LIMM Lethal Infantile Mitochondrial Myopathy; MMC Maternal Myopathy and Cardiomyopathy; NARP Neurogenic muscle weakness, Ataxia, and Retinitis Pigmentosa; Leigh Disease: FICP—Fatal Infantile Cardiomyopathy Plus, a MELAS-associated cardiomyopathy: MELAS Mitochondrial Encephalomyopathy with Lactic Acidosis and Stroke like episodes; LDYT Leber's hereditary optic neuropathy and Dystonia; MERRF Myoclonic Epilepsy and Ragged Red Muscle Fibers; MHCM Maternally inherited Hypertrophic Cardiomyopathy: CPEO Chronic Progressive External Ophthalmoplegia; KSS Kearns Sayre Syndrome; DM Diabetes Mellitus; DMDF Diabetes Mellitus+Deafness; CIPO Chronic Intestinal Pseudoobstruction with myopathy and Opthalmoplegia; DEAF Maternally inherited DEAFness or aminoglycoside-induced DEAFness; PEM Progressive encephalopathy; SNHL SensoriNeural Hearing Loss; Encephalomyopathy; Mitochondrial cytopathy: Dilated Cardiomyopathy: GER Gastrointestinal Reflux: DEMCHO Dementia and Chorea; AMDF Ataxia, Myoclonus; Exercise Intolerance: ESOC Epilepsy, Strokes, Optic atrophy, & Cognitive decline; FBSN Familial Bilateral Striatal Necrosis: FSGS Focal Segmental Glomerulosclerosis: LIMM Lethal Infantile Mitochondrial Myopathy; MDM Myopathy and Diabetes Mellitus: MEPR Myoclonic Epilepsy and Psychomotor Regression; MERME MERRF/MELAS overlap disease; MHCM Maternally Inherited Hypertrophic CardioMyopathy: MICM Maternally Inherited Cardiomyopathy; MILS Maternally Inherited Leigh Syndrome; Mitochondrial Encephalocardiomyopathy; Multisystem Mitochondrial Disorder (myopathy, encephalopathy, blindness, hearing loss, peripheral neuropathy); NAION Nonarteritic Anterior Ischemic Optic Neuropathy; NIDDM Non-Insulin Dependent Diabetes Mellitus; PEM Progressive Encephalopathy; PME Progressive Myoclonus Epilepsy; RTT Rett Syndrome: SIDS Sudden Infant Death Syndrome: MIDD Maternally Inherited Diabetes and Deafness; and MODY Maturity-Onset Diabetes of the Young, and MNGIE), and other metabolic diseases).

In one aspect, disclosed herein is a method of imaging a desired area in a subject, the method comprising administering to the subject a cyclic polypeptide comprising a sequence of X-Cys-Y-Cys-Z, wherein X is an amino acid sequence of 0-30 amino acids, Y is an amino acid sequence of 2-20 amino acids, Z is an amino acid sequence of 0-30 amino acids, and at least two Cys are cysteine amino acids; and wherein the cyclic polypeptide is operably linked to an imaging agent, wherein the imaging agent is targeted to the desired area and said desired area is imaged.

In some embodiments, the methods of any preceding aspect comprises X an as amino acid sequence of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 amino acids, Y as an amino acid sequence of 2, 3, 4, 5, 6, 7, 8, 9, 10, 11, 12, 13, 14, 15, 16, 17, 18, 19, 20 amino acids, and Z as an amino acid sequence of 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 amino acids.

In some embodiments, the desired area comprises a tumor. In some embodiments, the tumor is derived from the cancer of any preceding aspect. In some embodiments, the desired area comprises an infected tissue or organ. In some embodiments, the infected tissue or organ is caused by the disease or pathogen of any preceding aspect. In some embodiments, the desired area comprises a damaged, weakened, or dysfunctional tissue or organ. In some embodiments, the damaged, weakened, or dysfunctional tissue or organ is caused by the disease of any preceding aspect.

In some embodiments, the imaging agent is useful in magnetic resonance, or variations thereof including, but not limited to nuclear magnetic resonance imaging (NMRI), magnetic resonance tomography (MRT), functional MRI (fMRI), magnetic resonance venography (MRV), magnetic resonance angiography (MRA), and cardiac MRI. In some embodiments, the imaging agent comprises an iron oxide microparticle. In some embodiments, the imaging agent comprises a fluorescein molecule. In some embodiments, the fluorescein molecule comprises fluorescein isothiocyanate (FITC), succinimidyl ester modified fluorescein, carboxyfluorescein, carboxyfluorescein succinimidyl ester, pentafluorophenyl esters (PFP), tetrafluorophenyl esters (TFP), or other fluorescein derivatives. Other imaging modalities can be utilized herein including, but not limited to computed tomography (CT) scanning and X-ray imaging.

### Methods of Treating and/or Preventing Disease

In one aspect, disclosed herein is a method of treating a subject in need of therapeutic intervention, the method comprising administering to the subject a peptide-antibody complex comprising a cyclic polypeptide operably linked to an antibody or fragments thereof, wherein the cyclic polypeptide comprises an amino acid sequence of X-Cys-Y-Cys-Z, wherein X is an amino acid sequence of 0-30 amino acids, Y is an amino acid sequence of 2-20 amino acids, Z is an amino acid sequence of 0-30 amino acids, and at least two Cys are cysteine amino acids; wherein the antibody or fragments thereof comprise a single chain variable fragment (scFv); and wherein the cyclic polypeptide improves binding of the antibody or fragments to an antigen.

In some embodiments, the method comprises an amino acid sequence, X, comprising 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 amino acids. In some embodiments, the method comprises an amino acid sequence, Y, comprising 2, 3, 4, 5, 6, 7, 8, 9, 10, 11, 12, 13, 14, 15, 16, 17, 18, 19, 20 amino acids. In some embodiments, the method comprises an amino acid sequence, Z, comprising 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 amino acids. In some embodiments, the therapeutic intervention comprises the cyclic polypeptide. In some embodiments, the therapeutic intervention comprises the cyclic polypeptide and the functional molecule In some embodiments, the cyclic polypeptide alone or in combination with the functional molecule is further combined with another therapeutic composition including, but not limited to an antibiotic, an anti-inflammatory compound, a sedative, an anesthetic, an anti-septic agent, and an immunosuppressive agent.

In some embodiments, the method comprises an antibody or fragments thereof. In some embodiments, the method comprises a single chain variable fragment (scFv). In some embodiments, the method comprises a functional molecule as a linker when a heavy chain of the scFv is on one side of the linker and a light chain of the scFv is on the other side of the linker.

In some embodiments, the method treats and/or prevents one or more diseases of any preceding aspect.

In some embodiments, the method of any preceding aspect comprises a cyclic polypeptide comprising at least 80% sequence identity to SEQ ID NO: 21. In some embodiments, the method comprises a cyclic polypeptide comprising 80, 81, 82, 83, 84, 85, 86, 87, 88, 89, 90, 91, 92, 93, 94, 95, 96, 97, 98, or 99% sequence identity to SEQ ID NO: 21. In some embodiments, the method comprises a cyclic polypeptide comprising SEQ ID NO: 21. In some embodiments, the cyclic polypeptide of any preceding aspect recognizes an epitope.

### Methods of Administering Cyclic Polypeptides Compositions

In one aspect, disclosed herein are methods of administering cyclic polypeptide compositions of any preceding aspect for the use of detecting, identifying, diagnosing, treating, and/or preventing disease in a subject.

In one aspect, disclosed herein is a method of administering cyclic polypeptide compositions of any preceding aspect for the use of imaging a desired area in a subject.

The cyclic polypeptide composition may be administered in such amounts, time, and route deemed necessary in order to achieve the desired result. The exact amount of the cyclic polypeptide composition will vary from subject to subject, depending on the species, age, and general condition of the subject, the severity of the disease, the particular cyclic polypeptide composition, its mode of administration, its mode of activity, and the like. The cyclic polypeptide composition is preferably formulated in dosage unit form for ease of administration and uniformity of dosage. It will be understood, however, that the total daily usage of the cyclic polypeptide composition will be decided by the attending physician within the scope of sound medical judgment. The specific therapeutically effective dose level for any particular subject will depend upon a variety of factors including the symptoms being treated and the severity of the disease; the activity of the cyclic polypeptide composition employed; the specific cyclic polypeptide composition employed; the age, body weight, general health, sex and diet of the patient; the time of administration, route of administration, and rate of excretion of the specific cyclic polypeptide composition employed; the duration of the treatment; drugs used in combination or coincidental with the specific cyclic polypeptide composition employed; and like factors well known in the medical arts.

The cyclic polypeptide composition may be administered by any route. In some embodiments, the cyclic polypeptide composition is administered via a variety of routes, including oral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, buccal, enteral, sublingual; by intratracheal instillation, bronchial instillation, and/or inhalation; and/or as an oral spray, nasal spray, and/or aerosol. In general, the most appropriate route of administration will depend upon a variety of factors including the nature of the cyclic polypeptide composition (e.g., its stability in the environment of the subject's body), the condition of the subject (e.g., whether the subject is able to tolerate oral administration), etc.

The exact amount of cyclic polypeptide composition required to achieve a therapeutically or prophylactically effective amount will vary from subject to subject, depending on species, age, and general condition of a subject, severity of the side effects, identity of the particular compound(s), mode of administration, and the like. The amount to be administered to, for example, a child or an adolescent can be determined by a medical practitioner or person skilled in the art and can be lower or the same as that administered to an adult.

In one aspect, disclosed herein is a cyclic polypeptide composition of any preceding aspect and a pharmaceutically acceptable carrier selected from an excipient, a diluent, a salt, a buffer, a stabilizer, a lipid, an emulsion, a nanoparticle, and a cream. One or more active agents (e.g. cyclic polypeptide) can be administered in the “native” form or, if desired in the form of salts, esters, amides, prodrugs, or a derivative that is pharmacologically suitable. Salts, esters, amides, prodrugs, and other derivatives of the active agents can be prepared using standards procedures known to those skilled in the art of synthetic organic chemistry and described, for example, by March (1992) Advanced Organic Chemistry; Reactions, Mechanisms, and Structure, 4^{th} Ed. N. Y. Wiley-Interscience.

A number of embodiments of the disclosure have been described. Nevertheless, it will be understood that various modifications may be made without departing from the spirit and scope of the invention. Accordingly, other embodiments are within the scope of the following claims.

By way of non-limiting illustration, examples of certain embodiments of the present disclosure are given below.

### EXAMPLES

The following examples are set forth below to illustrate the compositions, devices, methods, and results according to the disclosed subject matter. These examples are not intended to be inclusive of all aspects of the subject matter disclosed herein, but rather to illustrate representative methods and results. These examples are not intended to exclude equivalents and variations of the present invention which are apparent to one skilled in the art.

### Example 1

### Cyclic Peptide Linkers

### Cyclic Linker Variant Cloning

Oligonucleotides (Sigma-Aldrich) were designed to contain the desired mutations at the selected residue positions. The variant inserts were individually cloned using PCR and were ligated into the engineered easy-to-clone vector between the Bsal cloning sites, transformed into DH10B electrocompetent cells, and confirmed by sanger sequencing (OSU-CCC).

To verify cyclization and the binding of the linkers, streptavidin agarose (Pierce) was packed into an empty Nap-5 column. A proof-of-concept study was done using Cys linker (positive control) and Gly linker (negative control). 0.1 mg/ml protein was added to the column and incubated for an hour at room temperature. The column was then washed with 10 ml PBS and the bound protein was eluted in three different condition (B-mercaptoethanol, and biotin, and protein L elution buffer).

2 μl of 5 mg/ml BSM was spotted onto a nitrocellulose membrane. Once dried, the membrane was incubated in blocking buffer (5% BSA), 50 μL of 0.2 mg/ml protein, and His-probe™-HRP Conjugate (ThermoFisher) at room temperature for 30 min, respectively. Between each step, the membrane was washed with PBS three times. Finally, the membrane was developed using H₂O₂/DAB.

As a proof-of-concept study, the streptavidin binding cyclic peptide, C-HPQGPP-C (SEQ ID NO: 1), was incorporated into 3E8, an scFv with affinity for siayl-Tn antigen, with G4S at each end in order to maintain 3E8 in a monomeric format. A negative control was designed by replacing cysteines with glycine to prevent it from cyclization. A streptavidin column was prepared to verify cyclization and the binding of Cys (positive control) and Gly (negative control) linkers.

It will be apparent to those skilled in the art that various modifications and variations can be made in the present disclosure without departing from the scope or spirit of the invention Other embodiments of the disclosure will be apparent to those skilled in the art from consideration of the specification and practice of the methods disclosed herein. It is intended that the specification and examples be considered as exemplary only, with a true scope and spirit of the invention being indicated by the following claims.

1. A peptide-antibody complex comprising a cyclic polypeptide operably linked to a single chain variable fragment (scFv), wherein the cyclic polypeptide comprises an amino acid sequence of X-Cys-Y-Cys-Z, wherein X is an amino acid sequence of 0-30 amino acids, Y is an amino acid sequence of 2-20 amino acids, Z is an amino acid sequence of 0-30 amino acids, and at least two Cys are cysteine amino acids.

2. The complex of claim 1, wherein the at least two Cys form a disulfide bridge.

3. The complex of claim 1, wherein the scFv comprises a heavy chain and a light chain.

4. The complex of claim 1, wherein the scFv is part of a bispecific antibody.

5. The complex of claim 1, wherein the scFV is operably linked to a C terminus, a N terminus, or both C and N termini of the cyclic polypeptide.

6. The complex of claim 1, wherein the cyclic polypeptide becomes a linker when the scFV is on both the N and C terminus of the cyclic polypeptide.

7. The complex of claim 1, wherein the cyclic polypeptide becomes a linker when the heavy chain of the scFv is on one side of the linker and the light chain of the scFv is on another side of the linker.

8. The complex of claim 1, wherein the scFv comprises a diabody or a tetrabody.

9. The complex of claim 1, wherein the cyclic polypeptide comprises at least 80% 90% sequence identity to SEQ ID NO: 21.

10. The complex of claim 1, wherein the cyclic polypeptide comprises SEQ ID NO: 21.

11. The complex of claim 2, wherein the disulfide bridge forms a loop or a circle within the complex.

12. The complex of claim 11, wherein the loop or the circle form spontaneously.

13. A synthetic nucleic acid encoding the cyclic polypeptide of claim 1.

14. A vector comprising the synthetic nucleic acid of claim 13.

15. A cell comprising the vector of claim 14.

16. A method of treating a subject in need of therapeutic intervention, the method comprising administering to the subject a peptide-antibody complex comprising a cyclic polypeptide operably linked to to a single chain variable fragment (scFv), wherein the cyclic polypeptide comprises an amino acid sequence of X-Cys-Y-Cys-Z, wherein X is an amino acid sequence of 0-30 amino acids, Y is an amino acid sequence of 2-20 amino acids, Z is an amino acid sequence of 0-30 amino acids, and at least two Cys are cysteine amino acids; wherein wherein the cyclic polypeptide improves binding of the scFv to an antigen.

17-42. (canceled)

43. A polypeptide comprising any one of SEQ ID NOS: 1-20.
